(19) United States
(12) Patent Application Publication
(10) Pub. No.: US 2011/0108031 A1
(43) Pub. Date: May 12, 2011

(54) HEATED CONDUIT FOR RESPIRATORY HUMIDIFICATION

(75) Inventors: Neil Alex Korneff, Diamond Bar, CA (US); Rebecca Ann Wilday, Anaheim, CA (US)

(73) Assignee: CareFusion 2200 Inc., San Diego, CA (US)

(21) Appl. No.: 12/616,395

(22) Filed: Nov. 11, 2009

(51) Int. Cl. A61M 16/16

(52) U.S. Cl. 128/203.27

(57) ABSTRACT

A conduit for carrying humidified gases includes a tube extending between a first end and a second end and a helical wire positioned in the tube. The helical wire is formed of a conductive core defining a shape of the helical wire. An electrical receptacle is positioned at the first end of the tube and electrically coupled to the communication ends of the helical wire.

### BACKGROUND

Respiratory humidification systems are used in providing respiratory therapy to a patient. In general terms, the system includes a ventilator, humidifier and patient circuit. The ventilator supplies gases to a humidification chamber coupled with the humidifier. Water within the humidification chamber is heated by the humidifier, which produces water vapor that humidifies gases within the chamber. From the chamber, humidified gases are then carried to the patient through the patient circuit. One or more conduits of the patient circuit may be heated to minimize condensation within the conduit.

One current heated conduit utilizes a helical heating wire formed from a thermoplastic material to retain a shape of the wire within the conduit. Due to variation of temperature for the conduit during packaging, shipment, set-up, and use, rigidity of the thermoplastic wire can vary drastically, leading to problems associated with utilization of the conduit. For example, upon set-up, the thermoplastic is generally quire rigid, causing difficulty in adjusting and maneuvering the conduit to a desired position.

In another wire design for a conduit, a thin, low resistance wire (e.g., copper) is wound around a nylon core and power is provided through the wire. However, manufacturability and reliability of this wire can lead to a lack of connection between the wire and a source of electricity providing current to the wire. In particular, the size of the thin wire can be difficult to work with. As a result, a lack of connection may occur during manufacturing, during shipping or during use. If a lack of connection occurs, a patient receives cool, dry air, instead of desired heated, humidified air.

### SUMMARY

Aspects of concepts presented herein relate to a conduit for carrying humidified gases. The conduit includes a tube extending between a first end and a second end and a helical wire positioned in the tube. The helical wire is formed of a conductive core, which, in one embodiment, defines a shape of the helical wire. An electrical receptacle is positioned at the first end of the tube and electrically coupled to the communication ends of the helical wire. In a further embodiment, a thermoset material can insulate the conductive core of the helical wire. Additionally, the helical wire can be coupled with the second end.

### BRIEF DESCRIPTION OF THE DRAWINGS

The accompanying drawings are included to provide a further understanding of embodiments and are incorporated in and constitute a part of this specification. The drawings illustrate embodiments and together with the description serve to explain principles of embodiments. Other embodiments and many of the intended advantages of embodiments will be readily appreciated as they become better understood by reference to the following detailed description. The elements of the drawings are not necessarily to scale relative to each other. Like reference numerals designate corresponding similar parts.

FIG. 1 is a schematic view of a respiratory humidification system.

FIG. 2 is a schematic side view of a heated conduit.

FIG. 3 is a side view of a device end of a heated conduit.

FIG. 4 is a side view of a patient end of a heated conduit.

FIG. 5 is a side view of an alternative helical shape for a wire within a heated conduit.

FIG. 6 is a segment of a wire having a stranded core.

FIG. 7 is a segment of a wire having a solid core.

FIGS. 8-10 are alternative connectors that can be positioned in a heated conduit.

### DETAILED DESCRIPTION

In the following Detailed Description, reference is made to the accompanying drawings, which form a part hereof, and in which is shown by way of illustration specific embodiments in which the invention may be practiced. In this regard, directional terminology, such as “top,” “bottom,” “front,” “back,” “leading,” “trailing,” etc., is used with reference to the orientation of the Figure(s) being described. Because components of embodiments can be positioned in a number of different orientations, the directional terminology is used for purposes of illustration and is in no way limiting. It is to be understood that other embodiments may be utilized and structural or logical changes may be made without departing from the scope of the present invention. The following detailed description, therefore, is not to be taken in a limiting sense, and the scope of the present invention is defined by the appended claims.

It is to be understood that the features of the various exemplary embodiments described herein may be combined with each other, unless specifically noted otherwise.

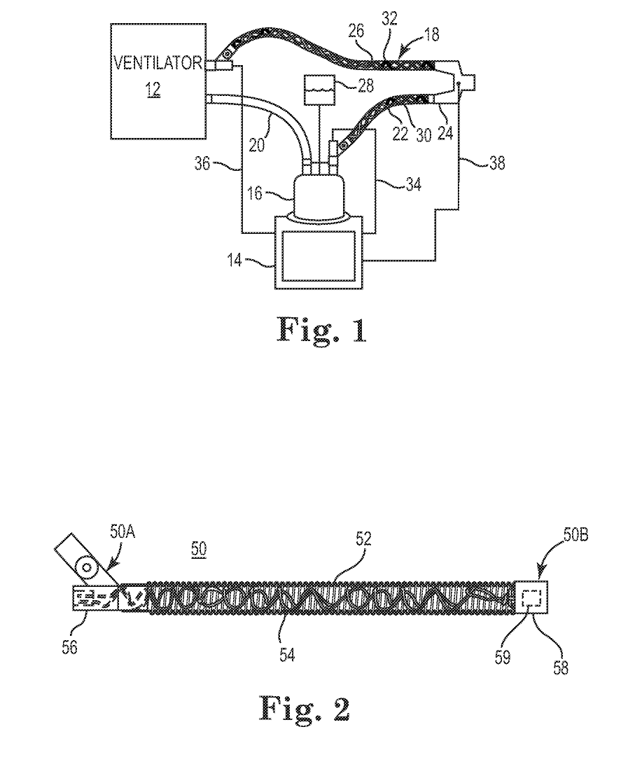

FIG. 1 is a schematic view of a respiratory humidification system 10 including a ventilator 12, humidifier 14 having a humidification chamber 16 and a patient circuit 18. It is worth noting that system 10 is one exemplary environment for concepts presented herein. For example, other forms of respiratory therapy can be used with the concepts presented herein such as a CPAP (Continuous Positive Airway Pressure) system or other system that may add or remove one or more of the components of system 10. Ventilator 12 supplies gases to humidification chamber 16 through an initial conduit 20. Humidifier 14 heats water within the chamber 16, which is then output to patient circuit 18. Patient circuit 18 includes an inspiratory conduit (or limb) 22, a y-connector 24 and an expiratory conduit (or limb) 26. In alternative embodiments, for example in a CPAP system, the y-connector 24 and/or expiratory conduit 26 can be eliminated. Inspiratory conduit 22 transmits humidified gases from chamber 16 to a patient through a y-connector 24. The y-connector 24 can be selectively coupled to a patient interface such as an endotracheal tube. Other patient interfaces can include masks, nasal prongs, etc. After breathing in the humidified gases, the patient can exhale, transmitting exhaled gases through expiratory conduit 26 back to ventilator 12. Liquid is supplied to the chamber 16 from a fluid source 28, which, in one embodiment comprises a bag of liquid (e.g., water) fluidly coupled to chamber 16. In one embodiment, chamber 16 can include a float valve system as described in co-pending U.S. application Ser. No. ______, entitled “Float Valve System For A Respiratory Humidification System,” filed on even date herewith, the contents of which are hereby incorporated in their entirety.

Inspiratory conduit 22 and expiratory conduit 26 include helical wires 30 and 32, respectively, positioned therein that, when heated, minimize condensation that may occur in the inspiratory conduit 22 or expiratory conduit 26. To minimize condensation, humidifier 14 supplies electrical power to helical wires 30 and 32 through electrical connectors 34 and 36, respectively. Helical wires 30 and 32 are selected with a desired resistance in order to heat humidified air within conduits 22 and 26, respectively, to a desired level. Additionally, humidifier 14 receives sensory inputs from a sensor input connector 38, which may provide temperature and/or flow information of gases within the patient circuit 18 so as to adjust power provided to helical wires 30 and 32. In one example, a temperature and/or flow sensor can be coupled to sensor input connector 38 to provide information indicative of temperature and/or flow within system 10. In one example, the sensor can be coupled to inspiratory limb 22 or y-connector 24.

FIG. 2 illustrates a conduit 50 having a first end (e.g., a device end) 50A and a second end (e.g., a patient end) 50B that can be utilized in respiratory humidification system 10. For example, conduit 50 is an exemplary embodiment of conduits 22 and 26 positioned in respiratory humidification system 10 of FIG. 1. If used as conduit 22 in FIG. 1, device end 50A would be positioned proximate chamber 16. If used as conduit 26 in FIG. 1, device end 50A would be positioned proximate ventilator 12. Conduit 50 further includes a tube 52, a helical wire 54 positioned within the tube 52, a first connector 56 positioned at first end 50A and a second connector 58 positioned at second end 50B. Connector 56 includes multiple branches, one for creating an electrical connection with wire 54 and one for fluid coupling to ventilator 12 or humidifier 14. Connector 58 is coupled with a branch of y-connector 24, proximate a patient. In one alternative embodiment, connector 58 can be equipped with one or more ports, one of which is schematically illustrated at port 59. The port 59 can be integrally formed in connector 58 or directly coupled to thereto. The port 59 can be equipped to receive a temperature probe, flow sensor, metered dose inhaler and/or combinations thereof. When port 59 is utilized to receive a temperature probe, it can be advantageous for maintaining consistent temperature of fluid within conduit 50 due to the fixed length of tube 52 in order to establish a consistent feedback loop to humidifier 14.

In the embodiment illustrated, tube 52 is a flexible corrugated tube adapted to be coupled between a device (e.g., ventilator 12, humidification chamber 16) and y-connector 24. In other embodiments, tube 52 need not be corrugated and can include various other textures and configurations. Wire 54 is formed of a conductive core insulated with a thermoset material (e.g., silicone) and, as discussed below, includes communication ends for electrically coupling wire 54 to humidifier 14 (e.g., through one of electrical connectors 34 and 36 of FIG. 1) and a loop portion coupled to connector 58. Thus, wire 54 is in fixed relation to connectors 56 and 58. In a further embodiment, wire 54 need not be fixed to connector 58, such that a clip or other mechanism can be used to secure wire 54 to tube 52 at a distance along conduit 50. In the embodiment illustrated, wire 54 is in the shape of a double helix including two helices that are out of phase approximately 180°. Although there are other ways to form wire 54, one approach is to hold the wire near a middle portion and spin the middle portion to create a helical shape. The core of the wire 54 provides a predetermined helical shape of the wire 54 such that the wire 54 is positioned proximate a circumference of tube 52, whereas a pitch of wire 54 is a function of a length for conduit 50, between ends 50A and 50B. For example, as a length of conduit 50 increases, the pitch of wire 54 will decrease and, as a length of conduit decreases, the pitch of wire 54 will increase. That is to say, the conductive core of wire 54 provides a shape that positions wire 54 near a circumference of tube 52 so as to provide desired heating of air within the tube 52 where air tends to cool and a pitch of the wire 54 is a function of the length of conduit 50 in a direction from end 50A to end 50B of conduit 50.

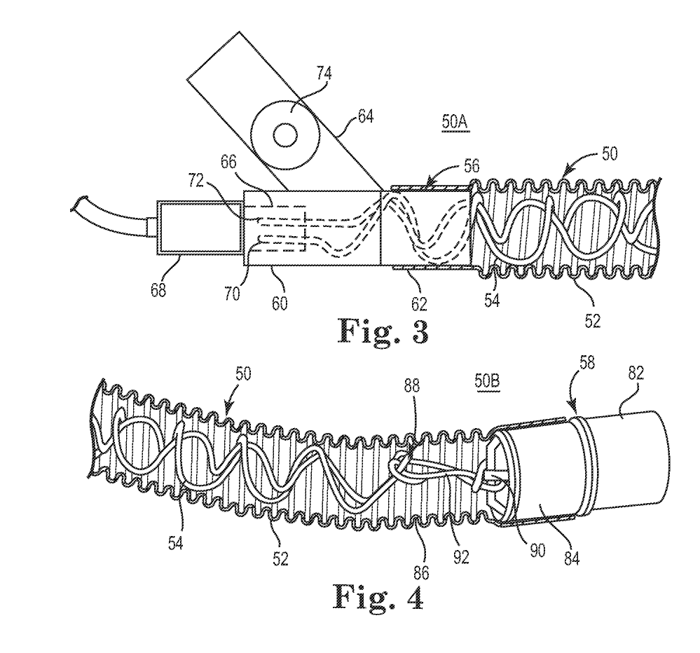

Turning to FIG. 3, connector 56 at first end 50A includes a first branch 60, a second branch 62, and a third branch 64, which is oriented transversely to first branch 60 and second branch 62. First branch 60 is coupled with tube 52 of conduit 50 whereas second branch 62 includes an electrical receptacle 66 that is coupable to an electrical connector 68. To provide current to wire 54, electrical connector 68 is electrically coupled to humidifier 14 (FIG. 1), which selectively delivers power to electrical connector 68. Helical wire 54 is plugged into receptacle 66 at communication ends 70 and 72, which can be a portion void of an insulating coating (e.g., a thermoset coating stripped from a wire core). Current is transmitted from humidifier 14 to connector 68, which in turn provides current to wire 54 through receptacle 66. Transverse branch 64 includes a sensor entry port 74 that can receive a flow and/or temperature sensor (e.g., for communicating measurements to humidifier 14 through sensor input connector 38 of FIG. 1). Additionally, third branch 64 can be fluidly coupled with ventilator 12 or chamber 16 either directly or through another conduit coupled to branch 64.

FIG. 4 is a view of a patient end 50B of conduit 50. If used as either conduit 22 or 26 of FIG. 1, device end 50B would be coupled with a branch of y-connector 24. The patient end 50B includes connector 58. Connector 58 includes a first branch 82 adapted for coupling to y-connector 24 and a second branch 84 for coupling to the tube 52 of conduit 50. Attached to branch 84 is a coupling mechanism 86 that extends from branch 84 and is secured to a loop portion 88 of helical wire 54. Coupling mechanism 86 includes a base 90 extending from branch 84 and a stem 92 coupled with the loop portion 88 and base 90. As a result, wire 54 is fixed to connector 58.

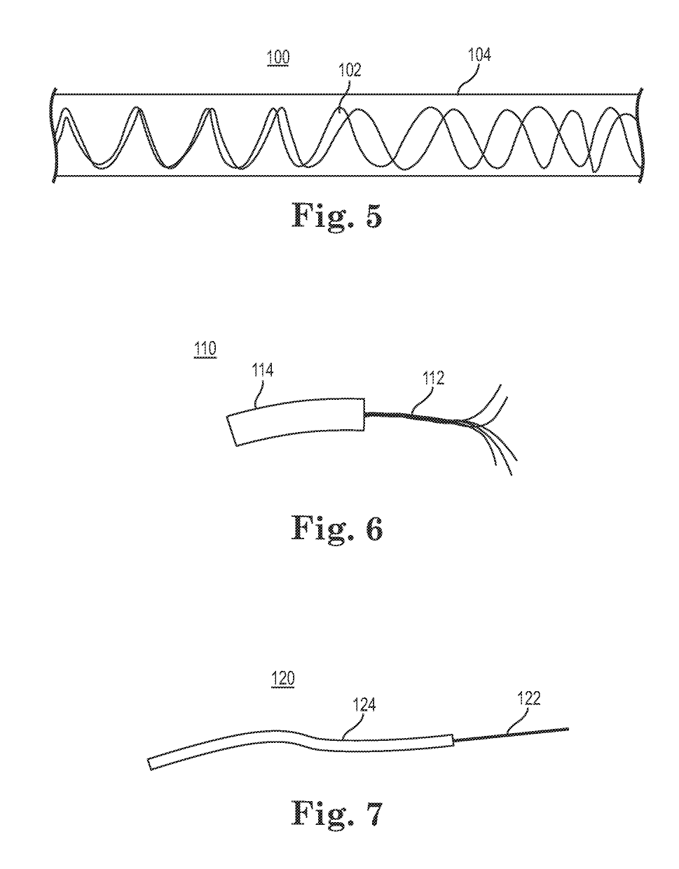

Conduit 50 can be modified in several different ways, as desired. For example, FIG. 5 illustrates an embodiment of an alternative conduit 100 having a wire 102 wound in a helical pattern within a tube 104. Similar to wire 54, wire 102 includes a conductive core surrounded by a thermoset material. However, in the embodiment illustrated, wire 102 is wound in a helical shape that is formed of two helices translated along an axis of the helix and substantially in phase, as opposed to the helical shape of wire 54 in FIG. 2, wherein two helices are provided out of phase about 180°. Additionally, tube 104 is smooth, in contrast to corrugated tube 52 of FIG. 2.

As discussed above, helical wires 54 and 102 are formed of a solid core surrounded by a thermoset material. The solid core is useful in easily forming an electrical connection with receptacle 66 (FIG. 3) as well as providing a shape of the helical wires with a desired flexibility. As a result, set-up of the conduit can be easily performed in a reliable manner. The thermoset insulating material is selected so as to not melt or burn during use of the wire. In FIG. 6, wire 110 includes a stranded wire core 112 that is surrounded by a thermoset insulating layer 114. In FIG. 7, wire 120 includes a solid core 122 surrounded by a thermoset insulating layer 124. Example materials for core 112 and core 122 include, but are not limited to Alloy 433 (304 stainless steel; 23 gauge; resistance of 0.865 Ohms/foot), Alloy 294 (55% copper, 45% nickel; 23 gauge; resistance of 0.6 Ohms/foot) and Alloy 675 (60% nickel, 25% iron, 15% chrome (chromium); 25 gauge; resistance of 2.2 Ohms/foot).

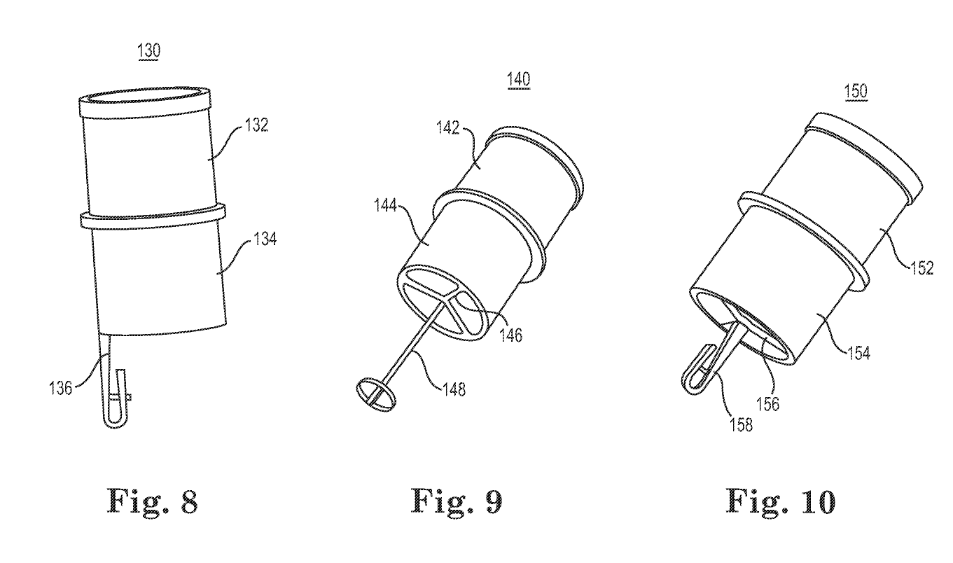

FIGS. 8-10 illustrate various connectors that can be used in place of connector 80 of FIG. 3. Each of the connectors include a stem for coupling to loop portion 88 of helical wire 54. FIG. 8 illustrates a connector 130 having a first branch 132. A stem 136 is coupled to branch 134 for coupling to loop portion 88 of wire 54. Similarly, FIG. 9 illustrates a connector 140 having a first branch 142 and a second branch 144. A base portion 146 and stem 148 are used to couple wire 54 to connector 140. FIG. 10 illustrates a connector 150 with a first branch 152 and a second branch 154. A base 156 and stem 158 are provided for coupling wire 54 to connector 150.

Although specific embodiments have been illustrated and described herein, it will be appreciated by those of ordinary skill in the art that a variety of alternate and/or equivalent implementations may be substituted for the specific embodiments shown and described without departing from the scope of the present invention. This application is intended to cover any adaptations or variations of the specific embodiments discussed herein. Therefore, it is intended that this invention be limited only by the claims and the equivalents thereof.

What is claimed is:

1. A conduit for carrying humidified gases, comprising:
a tube extending between a first end and a second end;
a helical wire positioned in the tube and including two communication ends and a loop portion, the helical wire formed of a conductive core insulated with a thermoset material;
a first connector positioned at the first end of the tube and including an electrical receptacle connected to the communication ends of the helical wire; and
a second connector positioned at the second end of the tube and including a coupling mechanism integrally formed therein and coupled to the loop portion of the helical wire.

2. The conduit of claim 1, wherein the conductive core defines a shape of the helical wire.

3. The conduit of claim 1 wherein the conductive core comprises stranded wire.

4. The conduit of claim 1 wherein the conductive core comprises a single solid wire.

5. The conduit of claim 1 wherein the thermoset material is silicone.

6. The conduit of claim 1 wherein the tube is corrugated.

7. The conduit of claim 1 wherein the first connector includes a first branch coupled with the tube, a second branch housing the electrical receptacle and a third branch oriented transverse to the first and second branch for carrying gases.

8. The conduit of claim 1, wherein the communication ends include a portion void of thermoset material and positioned in the electrical receptacle.

9. The conduit of claim 1, wherein the second connector includes a port configured to receive a temperature sensor.

10. The conduit of claim 1, wherein the second connector includes a port configured to receive a metered dose inhaler.

11. A conduit for carrying humidified gases, comprising:
a tube extending between a first end and a second end;
a helical wire positioned in the tube and including two communication ends, the helical wire formed of a conductive core defining a shape of the helical wire; and
an electrical receptacle positioned at the first end of the tube and electrically coupled to the communication ends of the helical wire.

12. The conduit of claim 11 wherein the conductive core comprises stranded wire.

13. The conduit of claim 11 wherein the conductive core comprises a single solid wire.

14. The conduit of claim 11 wherein the thermoset material is silicone.

15. The conduit of claim 11 wherein the tube is corrugated.

16. The conduit of claim 11 wherein the helical wire is fixed to the first end and the second end of the tube.

17. The conduit of claim 11 wherein the predetermined shape of the helical wire is positioned proximate a circumference of the tube.

18. A circuit for a respiratory humidification system, comprising:
an inspiratory conduit extending from a chamber end to a patient end, the inspiratory conduit having a first helical wire positioned therein and including a first conductive core defining a shape of the first helical wire;
an expiratory conduit extending from the patient end to a device end, the expiratory conduit having a second helical wire positioned therein and including a second conductive core defining a shape of the second helical wire;
a first electrical connector electrically coupled to the first helical wire; and
a second electrical connector electrically coupled to the second helical wire.

19. The circuit of claim 18 wherein the first conductive core and the second conductive core comprise stranded wire.

20. The circuit of claim 18 wherein the first conductive core and the second conductive core comprise a single solid wire.

21. The circuit of claim 18 wherein the thermoset material is silicone.

22. The circuit of claim 18 wherein the tube is corrugated.

23. The circuit of claim 18 and further comprising a y-connector coupled to the inspiratory conduit and the expiratory conduit.
